(19) United States
(12) Patent Application Publication
(10) Pub. No.: US 2003/0181987 A1
(43) Pub. Date: Sep. 25, 2003

(54) Replacement of bearing surfaces for hip prosthesis

(76) Inventor: Sarah Muirhead-Allwood, London (GB)

Correspondence Address: BURNS DOANE SWECKER & MATHIS L L P, POST OFFICE BOX 1404, ALEXANDRIA, VA, 22313-1404, US

(21) Appl. No.: 10/258,820

(22) PCT Filed: Jun. 26, 2001
(86) PCT No.: PCT/GB01/02863

(30) Foreign Application Priority Data
Jun. 28, 2000 (GB) 0015855.0

(51) Int. Cl. A61F 2/32

(52) U.S. Cl. 623/22.15; 623/22.42; 623/22.43; 623/22.45

(57) ABSTRACT

A method of restoring a hip replacement prosthesis is described wherein the prosthesis comprises a stem, a femoral head and an acetabular shell having a polyethylene liner, the liner having become worn or damaged. The method comprises removing the liner and replacing it with a hard surface liner, securing it to the acetabular shell, the hard surface liner being shaped to fit the femoral head. The method is carried out without disrupting the fixing of the stem and/or the acetabular shell.

This invention relates to a method of restoring a hip replacement prosthesis and improved components for such a prosthesis.

The most commonly used method for the treatment of the arthritic hip is total hip replacement. This procedure involves fitting an acetabular component to the pelvic bone in place of the natural socket, and a femoral component which is fixed to the proximal end of the femur using an intramedullary stem, after resection of the diseased femur. Normally, the two components of the prosthesis may be cemented into position using acrylic bone cement or, particularly in the case of the femoral stem, may rely on bone attachment or ingrowth for anchorage into the natural bone. The femoral component includes a femoral head comprising a ball which articulates in a liner fitted into the acetabular component. The liner is conventionally formed from an ultra-high molecular weight polyethylene polymer and in some designs, the femoral head is exchangeable so that the surgeon can select a head appropriate in size for a particular patient.

In a number of case, the hip replacement prosthesis fails after a time. This is sometimes caused by aseptic loosening. Aseptic loosening results in bone resorption at the interface between the bone and the prosthesis and subsequent soft tissue formation leading to an unstable fixation. Aseptic loosening can be caused by wear particles generated by movement of the articulating surfaces. These wear particles may be small polyethylene particles less than 1 micron in size derived from the polyethylene liner.

The rate of wear of polyethylene liners tends to be greater in the case of small femoral heads which are used in small patients, including children. This arises from higher loads on the articulating surfaces and a higher rate of penetration of the femoral head into the polyethylene sockets. For such patients there is a high incidence of failure leading to one or more revision operations being necessary to replace the original prosthesis.

According to current practice where aseptic loosening has not occurred, revision operations involve replacing the acetabular liner and/or the femoral head. While this improves the condition in the short term, it does not prevent it recurring in the long term.

According to one aspect of the present invention there is provided a method of restoring a hip replacement prosthesis comprising a stem, femoral head and an acetabular shell having a polyethylene liner which has become worn, said method comprising removing the liner and replacing it with a hard surface liner, the liner being shaped to fit the femoral head, and securing the liner to the shell. The liner conveniently has a hard, wear-resistant surface for contacting the femoral head. Suitable materials include metals and ceramics or composite materials. For example, the hard surface liner may comprise a cobalt chrome alloy or a ceramic material such as aluminium oxide and/or zirconium oxide ceramic or glass material.

The femoral head may also be replaced by a substitute head shaped to fit the metal liner. This may be desirable, as after removal of the polyethylene liner, the space within the acetabular shell is generally different. In order to conveniently accommodate replacement of femoral heads of different sizes, the femoral component may comprise a modular head fitted to the stern, e.g. by means of a taper. The femoral head may include a tapered socket and in order to accommodate modular heads having sockets of varying tapers, a modular sleeve may be interposed between the femoral stem and the modular head.

The metal replacement liner is conveniently fixed into the acetabular shell by means of a cement, e.g. an acrylic bone cement.

The procedure described above enables a hip replacement prosthesis to be repaired or refurbished without disrupting the initial fixation of the step and/or acetabular components into the patient's bone.

In some circumstances, it may be desirable to cement the metal liner into the shell over an intermediate spacer, e.g. of polyethylene. This is desirable, for example, if the space within the shell cannot be easily accommodated by selecting a femoral head of appropriate size. The liner may be cemented directly over the existing polyethylene liner, particularly where this is in reasonably good condition. In other circumstances, the new intermediate liner may be placed inside the shell prior to fixing the metal liner, or the intermediate liner may be part of the hard surface liner, e.g. laminated or otherwise secured to the surface intended to contact the acetabular shell.

The exchange procedure preferably utilises existing off-the-shelf components already manufactured by orthopaedic equipment manufacturers, or would utilise modular devices specifically made for the purposes of the invention.

There are a number of different tapers used by orthopaedic companies for the modular femoral head and neck junctions. In some instances, this necessitates adapting the taper onto which the head fits. This could be achieved using a metal sleeve which would fit onto the taper of the femoral stem and would be adapted to fit the taper in the socket of the replacement femoral head.

By using this kind of sleeve, a number of different hip stems with different tapers could be adapted for use with different femoral heads. A number of different sleeves may have to be produced to cover the variability of different tapers used by different orthopaedic manufacturers. One particular advantage of the prosthesis of the invention is that it would prevent wear of polyethylene liners in patients of small stature occurring. In the long term, this would be a cost-effective procedure, as it would reduce the number of revision procedures that such patients require.

The invention is illustrated by the accompanying informal drawings, in which:

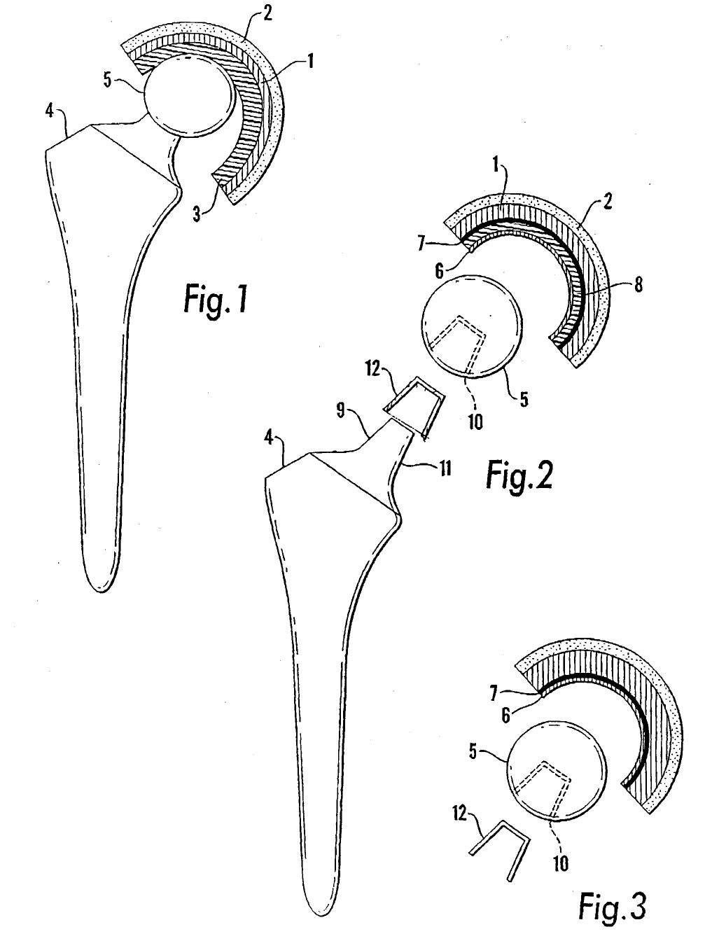

FIG. 1 is a perspective view of a femoral stem articulating within a worn acetabular ultra-high molecular weight polyethylene liner;

FIG. 2 is a similar view but showing, in exploded form, a femoral head on a femoral stem and a metal liner fitted to an acetabular shell; and

FIG. 3 shows a femoral head being introduced into an acetabular shell fitted with a cemented metal liner.

Referring to the drawings, the acetabular shell (1) is fixed into pelvic bone (2) by a cement-less fixing in which bone growth has been stimulated to engage the shell (1). This can be done, for example, by close fitting of the shell into its shaped socket in the pelvic bone and forming the surface in contact with the bone with micro-indentations and/or with an hydroxy apatite coating. The inner surface of the acetabular shell supports an ultra-high molecular weight polyethylene liner (3). A femoral component comprising a femoral stem (4) carries a femoral head (5) which articulates within the acetabular shell. As can be seen from the drawing, the polyethylene liner has worn unevenly and is of different thickness in different parts of the shell.

FIG. 2 shows the improvement in accordance with the invention in which an acetabular shell (1) is anchored securely into a socket (2) in a pelvic bone. However, in this case, in order to prevent uneven wear, a metal liner (6) is cemented into the shell (1) by a layer of acrylic bone cement (7). In order to accommodate the additional space within the shell, a layer of polyethylene (8) forms an intermediate spacer between the layer of cement and the metal shell.

In the case of metal liners, it is desirable for the femoral head (5) to fit securely and with limited tolerance within the semi-spherical socket formed by the metal liner (6). Therefore, it is within the scope of this invention to provide modular heads (5) for the femoral stem (4) which arc attached by means of a taper to the femoral neck (9). In order to accommodate tapers of different degree in the socket of the femoral head and conform with a different taper (11) on the femoral stem, a sleeve (12) may be provided having tapering surfaces which are different on the outer annular surfaces.

FIG. 3 shows a variation of the arrangement shown in FIG. 2, in which the metal liner (6) is directly cemented via a bone cement (7) to the shell (1).

1. A hip replacement prosthesis for use in the restoration of an existing prosthesis; said replacement prosthesis comprising:
a spherical femoral head (5) with a tapered aperture therein, al femoral stem (4) with a tapered neck (9) adapted to be received in the tapered aperture in the femoral head (5), a sleeve (12) disposed between the tapered neck (9) and the aperture in the femoral head (5) and an acetabular shell (1) for fitment in a socket in a pelvic bone, the shell (1) having a hard surface liner (6) made of metal and/or ceramic for receiving the femoral head (5).

2. A prosthesis according to claim 1, wherein the liner (6) is fixed to the shell (1) with a layer (7) of bone cement.

3. A prosthesis according to claim 1, wherein the liner (6) is fixed in the shell (1) with a layer (7) of bone cement and an intermediate spacer (8).

4. A prosthesis according to claim 3, wherein the intermediate spacer (8) is made from a plastics material.

5. A kit of parts for use in the prosthesis according to any one or more of the preceding claims, said kit of parts comprising a modular replaceable spherical femoral head (5) with a tapered aperture therein, a sleeve (12) for fitment in the aperture in the head (5) to adapt the head for fitment to a tapered neck (9) of a femoral stem and an acetabular shell (1) with a hard surface liner (6) of metal and/or ceramic adapted to receive the femoral head (5).
